(19) United States
(12) Patent Application Publication
(10) Pub. No.: US 2025/0306041 A1
(43) Pub. Date: Oct. 2, 2025

(54) METHODS FOR EVALUATION OF EXTRACELLULAR VESICLES

(71) Applicant: THE TEXAS A&M UNIVERSITY SYSTEM, College Station, TX (US)

(72) Inventor: Ryang Hwa Lee, College Station, TX (US)

(21) Appl. No.: 19/097,796

(22) Filed: Apr. 1, 2025

Related U.S. Application Data
(60) Provisional application No. 63/572,747, filed on Apr. 1, 2024.

(51) Int. Cl. G01N 33/74; A61K 9/50; A61K 38/18; A61K 38/19; C12N 15/113; C12Q 1/6876; G01N 33/68

(52) U.S. Cl. CPC G01N 33/74; A61K 9/5068; A61K 38/1841; A61K 38/191; C12N 15/113; C12Q 1/6876; G01N 33/6863; C12N 2310/141; C12Q 2600/178; G01N 2333/495; G01N 2333/525

(57) ABSTRACT

Provided are methods for evaluating therapeutic potency of extracellular vesicles (EVs) and methods for selecting a population of EVs for therapy by measuring amount of one or more of TGF-β1 (Transforming Growth Factor-β1), TSG-6 (Tumor Necrosis Factor-Stimulated Gene 6 protein), and let-7b-5p in the EVs. A method for treating a disease in a subject by measuring amount of one or more of TGF-β1, TSG-6, and let-7b-5p in EVs, isolating a portion of the EVs containing the one or more of TGF-β1, TSG-6, and let-7b-5p above a predetermined threshold, and administering a therapeutically effective amount of such EVs to the subject is also provided.

### CROSS-REFERENCE TO RELATED APPLICATIONS

This application claims the benefit of and priority to U.S. Provisional Application No. 63/572,747, filed on Apr. 1, 2024, which is incorporated by reference herein in its entirety.

### GOVERNMENT SUPPORT

This invention was made with government support under grant no. R01 EY029350 awarded by National Eye Institute of the U.S. National Institutes of Health. The government has certain rights in the invention.

### TECHNICAL FIELD

This disclosure is related generally to methods and compositions for evaluating functions and/or therapeutic efficacy of extracellular vesicles (EVs).

### BACKGROUND

EVs, such as EVs derived from mesenchymal stem/stromal cells (MSCs), mediate the immunomodulatory effects of the parent cells, such as MSCs. These MSCs can be widely isolated from various tissues including bone marrow, umbilical cord, and adipose tissue, with the potential for self-renewal and multipotent differentiation, and can be produced in large quantities for clinical applications. The immunomodulatory capabilities of EVs stem from the proteins and genetic materials they carry from parent cells. However, the cargo contents of EVs are significantly influenced by MSC tissues and donors, cellular age, and culture conditions, resulting in functional and therapeutic efficacy variations. MSC-EVs are a promising cell-free approach for treatment of a variety of diseases, such as autoimmune disorders, wound healing, fibrosis, and spinal injuries.

### SUMMARY

Disclosed herein are methods using bioassays and biomarkers to evaluate the therapeutic efficacy of EVs, such as isolated MSC-EVs in immunomodulation. The assays provided herein include an ELISA (enzyme-linked immunosorbent assay)-based assay to quantify levels of one or more of TGF-β1 (Transforming Growth Factor-β1), TSG-6 (Tumor Necrosis Factor-Inducible Gene 6 protein also known as TNF-Stimulated Gene 6 protein), and/or let-7b-5p in MSC-EVs, which enables quantitative validation of the immunomodulatory potency of MSC-EVs based on the TGF-β1, TSG-6, and/or let-7b-5p content per number of EV particles. The assays provided herein offer practical means to evaluate the therapeutic efficacy of EVs and are valuable tools for establishing acceptance/rejection criteria for EVs, such as MSC-EVs before in vivo administration.

In one aspect, provided herein is a method for evaluating therapeutic potency of EVs. The method includes measuring amount of one or more of TGF-β1, TSG-6, and let-7b-5p in the EVs, thereby evaluating therapeutic efficacy of the EVs. In some embodiments, the method includes measuring the amount of TGF-β1 or TSG-6 in the EVs. In some embodiments, the method includes measuring the amount of TGF-β1 and TSG-6 in the EVs.

In some embodiments, the amount of one or more of TGF-β1, TSG-6, and let-7b-5p is measured by ELISA and/or RT-PCR. In some embodiments, the EVs are derived from mesenchymal stem cells. In some embodiments, the EVs are derived from various culture conditions such as monolayer culture, microcarrier culture, or spheroid culture.

In one aspect, provided herein is a method for selecting a population of EVs for administration to a subject for therapy. The method includes measuring amount of one or more of TGF-β1, TSG-6, and let-7b-5p in the population of EVs, determining that the measured amount of one or more of TGF-β1, TSG-6, and let-7b-5p in the population of EVs is above a predetermined threshold, and selecting such population of EVs for therapy. In some embodiments, the method includes measuring the amount of TGF-β1 or TSG-6 in the EVs. In some embodiments, the method includes measuring the amount of TGF-β1 and TSG-6 in the EVs. In some embodiments, the amount of one or more of TGF-β1, TSG-6, and let-7b-5p is measured by ELISA and/or RT-PCR. In some embodiments, the predetermined threshold is 50 picogram (pg) of TGF-β1 in 1×10⁹ EVs. In some embodiments, the predetermined threshold is 100 pg of TGF-β1 in 1×10⁹ EVs. In some embodiments, the predetermined threshold is 10 pg of TSG-6 in 1×10⁹ EVs. In some embodiments, the predetermined threshold is 50 pg of TSG-6 in 1×10⁹ EVs.

In some embodiments, the population of EVs are derived from mesenchymal stem cells. In some embodiments, the EVs are derived from various culture conditions, such as monolayer culture, microcarrier culture, or spheroid culture. In some embodiments, the therapy includes immunomodulation. In some embodiments, the therapy includes treating immune-mediated diseases, such as autoimmune diseases and inflammatory diseases.

In one aspect, provided is a method for treating a disease in a subject. The method includes measuring amount of one or more of TGF-β1, TSG-6, and let-7b-5p in EVs, isolating a portion of the population of EVs containing the one or more of TGF-β1, TSG-6, and let-7b-5p above a predetermined threshold, and administering a therapeutically effective amount of such EVs to the subject, thereby treating the disease.

In some embodiments, the method includes measuring the amount of TGF-β1 or TSG-6 in the EVs. In some embodiments, the method includes measuring the amount of TGF-β1 and TSG-6 in the EVs. In some embodiments, the amount of one or more of TGF-β1, TSG-6, and let-7b-5p is measured by ELISA and/or RT-PCR.

In some embodiments, the predetermined threshold is 50 picogram (pg) of TGF-β1 in 1×10⁹ EVs. In some embodiments, the predetermined threshold is 100 pg of TGF-β1 in 1×10⁹ EVs. In some embodiments, the predetermined threshold is 10 pg of TSG-6 in 1×10⁹ EVs. In some embodiments, the predetermined threshold is 50 pg of TSG-6 in 1×10⁹ EVs. In some embodiments, the EVs are derived from mesenchymal stem cells. In some embodiments, the EVs are derived from various culture conditions such as monolayer culture, microcarrier culture, or spheroid culture.

In some embodiments, the disease is immune-mediated diseases, such as autoimmune diseases and inflammatory diseases. In some embodiments, the autoimmune disease can be autoimmune uveitis, type 1 diabetes, Sjögren's syndrome, rheumatoid arthritis, scleroderma, inflammatory bowel disease (Crohn's disease, ulcerative colitis), Lupus, multiple sclerosis, Psoriasis, Guillain-Barre syndrome, Hashimoto's thyroiditis, or alopecia areata. In some embodiments, the inflammatory disease can be a cornea wound, foot ulcers, osteoarthritis, brain trauma injury, Alzheimer, acute lung injury, acute respiratory distress syndrome (ARDS), sepsis, organ transplantation and Graft-versus-host disease (GvHD).

In some embodiments, the therapeutically effective amount of the EVs is administered to the subject orally, intravenously, topically, intranasally, intramuscularly, subcutaneously, intradermally, intraperitoneally, intrathecally, epidurally, or intraocularly.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Embodiments will be readily understood by the following detailed description in conjunction with the accompanying drawings. Embodiments are illustrated by way of example and not by way of limitation in the figures of the accompanying drawings.

FIGS. 1A-1K depict biopotency assays and predictive biomarkers to validate the potency of MSC-EVs for the treatment of experimental autoimmune uveoretinitis (EAU). FIG. 1A is a graphical representation of the IFN-γ levels as measured by ELISA with conditioned medium of splenocytes stimulated with anti-CD3/CD28 microbeads for 72 hours with or without ML-EVs (0.75 to 3×10⁹ particles/ml) derived from population doubling 15 (PD15) or PD40 #7012 MSCs or MC-EVs derived from PD15 #7012 MSCs. FIG. 1B is a graphical representation of the IFN-γ levels as measured by ELISA with conditioned medium of splenocytes stimulated with anti-CD3/CD28 microbeads for 72 hours with or without ML-EVs or MC-EVs (3×10⁹ particles/ml) derived from PD15 #6015 or PD15 #7052 MSCs. The splenocyte assays in FIGS. 1A and 1B were performed with experimental replicates (n=3 to 5) and data are presented in mean±SD. **p<0.01, ***p<0.005, ****p<0.0001, by one-way ANOVA with Dunnett's multiple comparison tests. FIGS. 1C and 1D present the correlation between the level of TGF-β1 (FIG. 1C) or let-7b-5p (FIG. 1D) in EVs derived from early- or late-passage MSCs expanded under monolayer cultures or microcarrier cultures or different donors and their capacity in suppressing IFN-γ in anti-CD3/CD28-splenocytes in vitro (reduction % of IFN-γ secretion compared to the positive control). Each dot represents a single experiment testing MSC-EVs with the splenocyte assay and each assay was performed with experimental replicates (n=3 to 5) as shown in FIG. 1A. FIG. 1E presents the correlation between the levels of TGF-β1 and let-7b-5p in MSC-EVs. FIGS. 1F-1K present simple logistic regression curves and ROC curves showing the predicted anti-inflammatory potency of MSC-EVs in inhibiting IFN-γ secretion in anti-CD3/CD28-splenocytes by 30% or 60% compared to the positive control group, based on the amount of TGF-β1 (FIGS. 1F-1H) or let-7b-5p (FIGS. 1I-1K) in MSC-EVs (1×10⁹). In all cases R is the Pearson's correlation co-efficient. Logistic regression analysis was performed in PRISM.

FIGS. 1L-IN demonstrate that the knockdown (KD) of let-7b in MSCs affects let-7b and TGF-β1 expression in MC-EVs. MSCs cultured on microcarriers were transfected with control or let-7b inhibitors (1 nM) and after 24 hrs, the transfected MSCs were used to produce MC-EVs. As shown in FIGS. 1L-1M, the let-7b expression levels in both MSCs and MC-EVs were confirmed by RT-qPCR. FIG. 1N is a graphical representation of the TGFβ1 expression, as measured by ELISAs, in control MC-EVs and let-7b KD MC-EVs. Data are presented in mean±SD. **p<0.01; ****p<0.001, by student t-test.

FIGS. 2A-2F demonstrate the effect of MSC-EVs carrying high levels of TGF-β1 in halting the disease progress in EAU mice. FIG. 2A illustrates the monolayer (ML) and microcarrier (MC) culture conditions for MSC-EV production. FIG. 2B is a graphical representation of the results of ELISA for TGF-β1 levels and RT-PCR for let-7b-5p levels in ML- or MC-EVs. ****p<0.001, by student t-test. FIG. 2C is a graphical representation of the results of the IFN-γ levels as measured ELISA with conditioned medium of splenocytes stimulated with anti-CD3/CD28 microbeads for 72 hours with or without ML-EVs or MC-EVs (1.5 to 3×10⁹ particles/ml). The splenocyte assay was performed with experimental replicates (n=3 to 5). **p<0.01, ****p<0.0001, by one-way ANOVA with Dunnett's multiple comparison tests. FIG. 2D is an illustration of the experimental scheme. On day 0, EAU was induced by SC injection of interphotoreceptor retinoid-binding protein (IRBP) and IP injection of Pertussis toxin. On day 14, ML- or MC-EVs (1.0×10¹⁰ EV particles/mouse) were injected into tail vein. As a control, the same volume of PBS was injected. On day 28, the eyeballs were collected for assays. FIG. 2E presents representative microphotographs of hematoxylin and eosin (H-E) staining of the eyes (100× magnification). FIG. 2F is a graphical representation of histological disease scores of retinal pathologies. Each dot represents a single animal, and data are presented in mean±SD. *p<0.05, **p<0.01, by one-way ANOVA with Dunnett's multiple comparison tests.

FIGS. 3A-3F present the effect of MSC-EVs in inhibiting T cell infiltration in EAU mice. FIG. 3A is a set of representative microphotographs of anti-CD3 immunostaining of the eyes (100× and 400× magnification). FIG. 3B is a graphical representation of the number of CD3⁺ cells in the retina and vitreous cavity (B). Each dot represents a single animal, and data are presented in mean±SD. *p<0.05, **p<0.01, ****p<0.0001 by one-way ANOVA with Dunnett's multiple comparison tests. FIG. 3C is a graphical representation of the results of the RT-PCR assay for IFN-γ mRNA levels with the left eyes of EAU mice at weeks 2, 3, and 4 following IRBP immunization. FIG. 3D presents the scatter plots of delta Ct (Δ Ct) mRNA levels in the left eyes of EAU mice or control mice vs. histological scores of the right eyes of the same mice where (r) is the Pearson correlation coefficient. FIG. 3E is a graphical representation of the results of the RT-PCR assays for IFN-γ and IL-17F mRNA levels with the left eyes of EAU mice (n=5 to 7) in FIG. 2F. *p<0.05, **p<0.01 by one-way ANOVA with Dunnett's multiple comparison tests.

FIGS. 4A-4E demonstrate the effect of MSC-EVs in suppressing the development of EAU in an adoptive transfer model of EAU. FIG. 4A illustrates an experimental scheme. Splenocytes were isolated from EAU mice and cultured with IRBP peptide 1-20 for 3 days and IBPR reactive T cells (4×10⁷ CD4+ T cells/mouse) were adoptively transferred into C57BL/6 mice by IP injection followed by IV injection of PBS, or ML or MC-EVs (1×10¹⁰ EV particles/mouse). On day 1 and 2, eye tissues were collected for RT-PCR assays in 4B-C and on day 14, eye tissues were collected for histology and RT-PCR assays in 4D-E. FIG. 4B is a graphical representation of the results of the RT-PCR assays for IFN-γ (left), IL-17F (middle) and TNF-α (right) levels in the eyes of recipient mice at 24 or 48 hours after adoptive transfer of IRBP-specific T cells (4×10⁷ cells/mouse; via IP). The IRBP-specific T cells were isolated from EAU mice, expanded with IRBP peptide 1-20 for 3 days. FIG. 4C is a graphical representation of the results of the RT-PCR assays for IFN-γ and IL-17F levels in the eyes of recipient mice at 48 hours after adoptive transfer of IRBP-specific T cells followed by IV injection of PBS or ML-EVs or MC-EVs (1×10¹⁰ EV particles/mouse; n=3 to 6 mice per group). *p<0.05; ****p<0.001; by one-way ANOVA with Dunnett's multiple comparison tests. FIG. 4D is a set of micrographs of H&E staining of the right eyes of retinal pathologies. FIG. 4E is a graphical representation of the results of the RT-PCR assays for IFN-γ. Each dot represents a single animal, and data are presented in mean±SD. *p<0.05, **p<0.01, ***p<0.005 by one-way ANOVA with Dunnett's multiple comparison tests.

FIGS. 5A-5D demonstrate the effect of MSC-EVs in suppressing the proliferation and inducing apoptosis in IRBP-stimulated splenocytes. FIG. 5A is a graphical representation of the results of the numbers of CD3+ cells (left) and the levels of IFN-γ as measured by ELISA (right). Splenocytes isolated from EAU mice were stimulated with IRBP (0.02 mg/ml) with or without ML-EVs or MC-EVs (1 to 3×10⁹ particles/ml) for 2 to 3 days. On day 3, floating cells were isolated for CD3+ cell counting, and conditioned medium was used for IFN-γ ELISA. FIGS. 5B and 5C present the cell cycle analysis of floating cells on day 2 for negative controls, positive controls, ML-EVs, and MC-EVs at G0/G1 and at G1, respectively. On day 2, viable T cells were collected using Lympholyte M from floating cells, incubated with or without ML-EVs or MC-EVs (3×10⁹ particles/ml) for 24 hours and analyzed for cell cycle. FIG. 5D is a set of Annexin V and PI staining results analyzed by flow cytometry, presenting the apoptosis analysis of floating cells on day 2.

FIGS. 6A-6G demonstrate the effect of MSC-EVs in suppressing the migration of activated T cells. FIG. 6A is a set of graphical representations of the results of the RT-PCR assays for different chemokine mRNA levels with the left eyes of mice on day 14 after CFA only or IRBP immunization. In FIGS. 6B and 6C, the top wells were loaded with IRBP reactive T cells (2.5×10⁵) from EAU mice with or without EVs (3×10⁹ particles/ml) and the lower chambers contained recombinant mouse CXCL9 (50 to 200 ng/ml) or IRBP peptides (250 to 1000 ng/ml). FIG. 6B is a graphical representation of the number of cells that migrated to the lower chamber after 3 hours. FIG. 6C is a graphical representation of the number of cells that migrated to the lower chamber after 6 h. *p<0.05; **p<0.01; ***p<0.005; ****p<0.001; by one-way ANOVA with Tukey's multiple comparison tests. In FIGS. 6D and 6E, the top wells were loaded with anti-CD3/CD28 microbead-stimulated T cells (2.5×10⁵) from naïve C57BL/6 mice and the lower chambers contained recombinant mouse CCL2 (FIG. 6D) or CCL19 (FIG. 6E) each at 20 ng/ml. After 3 or 6 hours of incubation at 37° C., the cells that migrated to the lower chamber were counted. ***p<0.005; ****p<0.001; by two-way ANOVA. In FIGS. 6F and 6G, the top wells were loaded with IRBP-specific T cells isolated from C57BL/6 mice with EAU (FIG. 6F) or B10 RIII mice with EAU (FIG. 6G) with or without MSC-EVs (1.5 to 3×10⁹ particles/ml) and the cells that migrated to the lower chamber were counted after 3 hours. ***p<0.005; by one-way ANOVA with Dunnett's multiple comparison tests.

FIGS. 7A-7H demonstrate the effect of MSC-EVs in suppressing the MAPK/ERK pathway in T cells. FIG. 7A is a photograph of the Western blot assay on naïve CD4⁺ T cells isolated from C57BL6 mice, and FIG. 7B is a graphical representation of the signal intensity of the bands in FIG. 7A. The CD4+ T cells were incubated with media containing CXCL19 (200 ng/ml) with or without MSC-EVs (0.75 to 3.0×10⁹ particles/ml) for 1 hour. FIGS. 7C, 7E, and 7G are photographs of Western blot assays on IRBP-specific T cells isolated from B10 RIII mice with EAU and FIGS. 7D, 7F, and 7H are graphical representations of the signal intensity of the bands in FIGS. 7C, 7E, and 7G, respectively. In FIGS. 7C and 7D, the IRBP-T cells were incubated with media containing CXCL19 (200 ng/ml) with or without MSC-EVs (3×10⁹ particles/ml) or rhTGF-β1 (1 ng/ml) for 1 hour. In FIGS. 7E and 7F, the IRBP-T cells were transiently transfected with control scrambled miRNA or let-7b-5p mimics (0.5 to 10 nM) and simultaneously stimulated with CXCL19 (200 ng/ml) with or without MSC-EVs (3×10⁹ particles/ml) for 1 hour. In FIGS. 7G and 7H, the IRBP-T cells were incubated with media containing CCL19 (20 ng/ml) with or without MSC-EVs (3×10⁹ particles/ml) or rhTGF-β1 (1 ng/ml) for 1 hour.

FIGS. 8A-8E provide a comparison of anti-inflammatory effects in MSC-EVs from monolayer (ML) and microcarrier (MC) Cultures. FIGS. 8A and 8B are graphical representations of the results from ELISAs for TGFβ1 and TSG-6 proteins in ML-EVs and MC-EVs. FIG. 8C is a graphical representations of the anti-inflammatory response of ML-EVs and MC-EVs in LPS-stimulated macrophages. Murine RAW 264.7 macrophage cells were stimulated with a dose of 10 ng/mL LPS with or without MSC-EVs. Cells or conditioned media were assayed at 6 and 24 hours after stimulation. FIG. 8C is a graphical representation of the assessment of IL-6 (left) and IL-10 (right) secretion in conditioned media 6 hours after stimulation and treatment with ML-EVs or MC-EVs at concentrations of 3×10⁹/mL or 6×10⁹/mL by ELISA. FIGS. 8D and 8E are graphical representations of the assessment of anti-inflammatory markers in macrophages 24 hours after stimulation and treatment with ML-EVs or MC-EVs at a concentration of 3×10⁹/mL. FIG. 8D is a graphical representation of the qPCR analysis of the gene expression of IL-6, IL-10, and Arg1. FIG. 8E is a graphical representation of the IL-6 and IL-10 secretion in conditioned media by ELISA.

FIG. 9 is a graphical representation of the comparison of anti-inflammatory effects in MC-EVs vs TSG-6 KD MC-EVs. Murine RAW 264.7 macrophage cells were stimulated with a dose of 10 ng/mL LPS with or without MSC-EVs (3×10⁹/mL). Assessment of IL-6 secretion in conditioned media 6 hours after stimulation by ELISA.

### DETAILED DESCRIPTION

Embodiments provided herein include assays using biomarkers to evaluate the therapeutic efficacy of MSC-EVs. EVs derived from MSCs have been recognized as a promising therapeutic for immune-mediated diseases as they exert immunomodulatory effects in several preclinical models, such as the immunomodulatory effects of MSC-EVs in autoimmune diseases, including experimental autoimmune uveoretinitis (EAU), type 1 diabetes and Sjögren's syndrome. MSC-EVs suppresses the development of T helper 1 (Th1) and Th17 cells as well as the activation of antigen presenting cells (APCs), thereby preventing the onset of autoimmune disease in these models. Moreover, MSC-EVs can target macrophages or induce regulatory T cells (Tregs), thereby suppressing inflammatory/immune responses.

Mechanistically, proteins and genetic materials, including microRNAs (miRNAs), carried by EVs from parent cells are responsible for the immunomodulatory effects of MSC-EVs. However, EVs undergo changes in tandem with their parent cells and due to this nature of EVs, their cargo contents are significantly influenced by several factors, such as MSC tissues and donors, cellular age, and culture conditions. It is well-known that the functional heterogeneity of MSCs poses a major challenge in developing rigorous and robust MSC therapy, given the substantial variations among MSC isolates due to differences in tissue sources, donors, and culture conditions. Consequently, EV-based therapies encounter the same challenge of functional variations as observed in MSC therapy. However, there are currently no reliable biopotency or surrogate assays available to validate the biological activity of MSC-EVs before in vivo administration.

One strategy to establish a surrogate assay for evaluating MSC-EV potency is to define effector molecules in MSC-EVs responsible for their therapeutic effects. EVs from early passage MSCs exhibited superior immunomodulatory potency compared to those from late-passage MSCs. Similarly, MSC-EVs generated under microcarrier culture conditions (MC-EVs) are more efficacious in reducing the inflammatory cytokine levels in LPS-challenged mice than those from MSC expanded under monolayers (ML-EVs). Moreover, MC-EVs suppress TLR4 (Toll-like receptor 4) or T cell receptor (TCR) downstream genes in LPS- or anti-CD3/CD28-stimulated splenocytes more effectively than ML-EVs. Further comparative molecular profiling analyses of MSC-EVs using proteomics and miRNA sequencing revealed that immunosuppressive factors, such as TGF-β1, TSG-6, and let-7b-5p (also referred to as let-7b), were enriched in MC-EVs compared to ML-EVs, and the levels of immunosuppressive factors were significantly reduced in EVs from late-passage MSCs. Importantly, TGF-β1, TSG-6, and let-7b-5p are key effectors in MSC-EVs via gain and loss-of-function studies of the target molecules in MSC-EVs. Provided herein are methods of predicting the immunomodulatory potency of MSC-EVs before in vivo administration using TGF-β1, TSG-6, and/or let-7b-5p as surrogate biomarker). The accuracy of the biomarkers in predicting the immunomodulatory potency of MSC-EVs was verified in murine models of EAU and additional in vitro bioassays reflect the Mode of Action (MoA) of MSC-EVs in vivo.

An “effective amount” or “therapeutically effective amount” is an amount sufficient to effect desired results (such as desired clinical results, to achieve therapeutic efficacy). A therapeutically effective dose can be administered in one or more administrations. “Administering” refers to the physical introduction of EVs to a subject in need thereof. Exemplary routes of administration for EVs, include intravenous, intranasal, intramuscular, subcutaneous, intraperitoneal, spinal or other parenteral routes of administration, for example by injection or infusion.

EVs may be administered via a non-parenteral route, or orally. Non-parenteral routes include by injection, such as intravenous, intranasal, intramuscular, intraarterial, intrathecal, intralymphatic, intralesional, intracapsular, intravitreal, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation and topical administration. Administering can also be performed, for example, once, a plurality of times, and/or over one or more extended periods. Therapeutic agents can be constituted in a composition, such as a pharmaceutical composition containing EVs and a pharmaceutically acceptable carrier. As used herein, a “pharmaceutically acceptable carrier” includes any and all solvents, dispersion media, coatings, antibacterial and antifungal agents, isotonic and absorption delaying agents, and the like that are physiologically compatible.

A “subject” refers an animal, such as a mammal, including a primate (such as a human, a non-human primate, such as a monkey) and a non-primate (such as a mouse). In some aspects of the disclosure, the subject is a human. In some aspects, the subject is a pediatric subject, such as a neonate, an infant, or a child. In other aspects, the subject is an adult subject.

As used herein, the terms “treating,” “treatment” and the like shall include the management and care of a subject or patient for the purpose of combating a disease, condition, or disorder and includes the administration of a composition to prevent the onset of the symptoms or complications, alleviate the symptoms or complications, reduce at least one associated sign, symptom, or condition, or eliminate the disease, condition, or disorder. Treatment also refers to a prophylactic treatment, such as prevention of a disease (such as autoimmune disease) or prevention of at least one sign, symptom, or condition associated with the disease. Treatment can also mean prolonging survival as compared to expected survival in the absence of treatment.

As used herein with respect to a parameter, the term “decreased” or “decreasing” or “decrease” or “reduced” or “reducing” or “reduce” or “lower” refers to a detectable (such as at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%) negative change in the parameter from a comparison control, such as an established normal or reference level of the parameter, or an established standard control. Accordingly, the terms “decreased,” “reduced,” and the like encompass both a partial reduction and a complete reduction compared to a control.

As used herein with respect to a parameter, the term “increased” or “increasing” or “increase” or “enhanced” or “enhancing” or “enhance” refers to a detectable (such as at least about 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, or 1000%) positive change in the parameter from a comparison control, such as an established normal or reference level of the parameter, or an established standard control.

The present disclosure describes various embodiments related to compositions and methods for evaluating therapeutic efficacy of EVs, selecting a population of EVs for therapy, or treating a disease in a subject using the EVs. In the following description, numerous details are set forth in order to provide a thorough understanding of the various embodiments. Before the present methods and compositions are described, it is to be understood that these embodiments are not limited to particular methods or compositions described, as such may, of course, vary. It is also to be understood that the terminology used herein is for the purpose of describing particular embodiments only, and is not intended to be limiting, as the scope of the present embodiments will be limited only by the appended claims. The description may use the phrases “in certain embodiments,” “in various embodiments,” “in an embodiment,” or “in embodiments,” which may each refer to one or more of the same or different embodiments. Furthermore, the terms “comprising,” “including,” “having,” and the like, as used with respect to embodiments of the present disclosure, are synonymous.

Provided herein are biopotency and surrogate assays for validating the therapeutic potency of EVs, such as MSC-EVs in vivo, which enable development of EV-based therapeutics for clinical applications. The biopotency assays provided herein are tailored to specific target diseases and reflect the MoA of MSC-EVs. Based on FDA guidelines, in vivo animal studies, in vitro organ, tissue or cell culture systems, or any combination of these can be used as biopotency assays and non-biological analytical assay(s) that indicates EV biological activity can be used a surrogate assay (FDA guidelines).

In one aspect, provided herein is a method for evaluating therapeutic potency of EVs. The method includes measuring amount of one or more of TGF-β1, TSG-6, and let-7b-5p in the EVs, thereby evaluating therapeutic efficacy of the EVs.

In another aspect, provided herein is a method for selecting a population of EVs for therapy. The method includes measuring amount of one or more of TGF-β1, TSG-6, and let-7b-5p in the population of EVs, determining that the measured amount of one or more of TGF-β1, TSG-6, and let-7b-5p in the population of EVs is above a predetermined threshold, and selecting such population of EVs for therapy. The amount of TGF-β1, TSG-6, and/or let-7b-5p can be measured by ELISA and/or RT-PCR.

The EVs can be derived from mesenchymal stem cells. The EVs can be derived from cells in monolayer (ML) culture or microcarrier (MC) culture. Without wishing to be bound by theory, the EVs derived from MC culture can contain higher amount of TGF-β1, TSG-6, and/or let-7b-5p as compared to the EVs derived from ML culture, and therefore, can be more therapeutically effective.

The predetermined threshold can be 50 pg of TGF-β1 in 1×10⁹ EVs. The predetermined threshold can be 100 pg of TGF-β1 in 1×10⁹ EVs. In certain embodiments, the predetermined threshold can range from 40 pg to 300 pg of TGF-β1 in 1×10⁹ EVs. For example, 1×10⁹ EVs carrying more than 50 pg and more than 100 pg of TGF-β1 can suppress 30% and 60% of IFN-γ, respectively, in anti-CD3/CD28-stimulated splenocytes. Let-7b-5p levels in EVs can discriminate EV potency to suppress 60% of IFN-γ secretion in anti-CD3/CD28-stimulated splenocytes with 100% sensitivity and 100% specificity. In some embodiments, the predetermined threshold is 10 pg of TSG-6 in 1×10⁹ EVs. In some embodiments, the predetermined threshold is 50 pg of TSG-6 in 1×10⁹ EVs. In certain embodiments, the predetermined threshold can range from 5 pg to 100 pg of TSG-6 in 1×10⁹ EVs. The therapy can include immunomodulation. The therapy can be directed to treating disease, such as an autoimmune disease and/or an inflammatory disease.

As the cytotoxic destruction of an organ by autoreactive Th1 and Th17 cells is the key pathological feature of autoimmune diseases, a disease-specific potency bioassay of MSC-EVs for the treatment of autoimmune diseases provided herein reflects the capacity to suppress Th1/Th17 cells. For example, the therapeutic potency of MSC-EVs in a murine model of ocular Sjögren's syndrome is strongly correlated with their potency in suppressing Th1/Th17 cytokines in TCR-stimulated splenocytes in vitro and further identified TGF-β1 and let-7b-5p as key effectors in MSC-EVs responsible for the suppression of Th1/Th17 cells in vitro. The biopotency assay provided herein enable the quantitative evaluation of the immunomodulatory potency of MSC-EVs generated under various conditions. The methods provided herein are based on measurements of one or more of TGF-β1, TSG-6, and let-7b-5p biomarker levels in MSC-EVs that provide characteristic data of MSC-EVs, and a correlation between the capacity of EVs to suppress the secretion of IFN-γ in TCR-stimulated splenocytes and the levels of biomarkers in MSC-EVs established based on the characteristic data. This correlation analysis defines a minimal concentration of TGF-β1 in MSC-EVs that effectively suppresses IFN-γ secretion in TCR-stimulated splenocytes, leading to the development of a surrogate assay with simple TGF-β1 ELISA for MSC-EVs. The surrogate assay provided herein serves as a valuable tool for establishing acceptance/rejection criteria for MSC-EVs before in vivo administration. In addition, the assay enables quantitative validation of the immunomodulatory potency of MSC-EVs based on the TGF-β1 content per number of EV particles, thus it offers a practical means to evaluate the therapeutic efficacy of EVs, circumventing labor-intensive cell cultures and animal testing. This approach can facilitate the optimization of upstream and downstream processing conditions for MSC-EVs, including aspects such as MSC culture conditions, culture media, donors, and EV isolation methods.

Additionally, the surrogate assay provided herein would help minimize the impact of functional variations between MSC-EV batches, thereby enhancing research rigor and reproducibility in MSC-EV studies. This advancement is pivotal in establishing robust MSC-EV therapeutics for treating autoimmune diseases. Further, the surrogate assay provided herein can evaluate MSC-EVs obtained from different tissues and help validate the biological function of MSC-EVs for various clinical applications.

Provided herein are methods for treating a disease in a subject. The method includes measuring amount of one or more of TGF-β1, TSG-6, and let-7b-5p in extracellular vesicles, isolating a portion of the EVs containing the one or more of TGF-β1, TSG-6, and let-7b-5p above a predetermined threshold, and administering a therapeutically effective amount of the EVs to the subject, thereby treating the disease. The amount of one or more of TGF-β1, TSG-6, and let-7b-5p can be measured by ELISA and/or RT-PCR.

The predetermined threshold can be 50 pg of TGF-β1 in 1×10⁹ EVs. The predetermined threshold is 100 pg of TGF-β1 in 1×10⁹ EVs. For example, 1×10⁹ EVs carrying more than 50 pg and more than 100 pg of TGF-β1 can suppress 30% and 60% of IFN-γ, respectively, in anti-CD3/CD28-stimulated splenocytes. Let-7b-5p levels in EVs can discriminate EV potency to suppress 60% of IFN-γ secretion in anti-CD3/CD28-stimulated splenocytes with 100% sensitivity and 100% specificity. In some embodiments, the predetermined threshold is 10 pg of TSG-6 in 1×10⁹ EVs. In some embodiments, the predetermined threshold is 50 pg of TSG-6 in 1× 10⁹ EVs.

The EVs can be derived from mesenchymal stem cells. The EVs can be derived from cells in monolayer culture or microcarrier culture or spheroid culture. The disease can be immune-mediated diseases, such as an autoimmune disease and/or an inflammatory disease. The autoimmune disease can be autoimmune uveitis, type 1 diabetes, Sjögren's syndrome, rheumatoid arthritis, scleroderma, inflammatory bowel disease (Crohn's disease, ulcerative colitis), Lupus, multiple sclerosis, Psoriasis, Guillain-Barre syndrome, Hashimoto's thyroiditis and alopecia areata. The inflammatory disease can be a cornea wound, a foot ulcer, osteoarthritis, brain trauma injury, Alzheimer, acute lung injury, acute respiratory distress syndrome, sepsis, organ transplantation and Graft-versus-host disease.

The therapeutically effective amount of the EVs can be administered to the subject in any suitable route. For example, the therapeutically effective amount of the EVs can be administered to the subject orally, intravenously, intranasally, intramuscularly, subcutaneously, intradermally, intraperitoneally, intrathecally, topically, intravitreally, epidurally, or intraocularly. Further, the therapeutically effective amount of the EVs can be administered to the subject in any suitable dosing regimen.

The accuracy of the surrogate assays provided herein is further validated through in vivo and in vitro potency assays that reflect the MoA of MSC-EVs in vivo. An adoptive transfer model of EAU is a useful model to demonstrate the inhibitory effect of MSC-EVs on autoreactive T cells. Upon adoptive transfer of retina-reactive T cells, they rapidly migrated into the eyes within a few days, causing retinal destruction in recipient mice. However, when co-administered with MSC-EVs, a significant reduction in the severity of EAU was observed, indicating the inhibitory effect of MSC-EVs on the infiltration of autoreactive T cells. Also, Th1 and Th17 cytokine levels in the eyes of recipient mice can reflect the number of migrated retina-reactive T cells, hence the inhibitory effect of MSC-EVs on autoreactive T cells in vivo can be quantitatively measured by RT-PCR assays for Th1 and Th17 cytokine levels in the eyes. Additionally, in vitro assays further revealed the MoA of MSC-EVs on the inhibition of activated T cell infiltration into the eyes. MSC-EVs may induce apoptosis, inhibit proliferation or/and suppress the migration of retina-reactive T cells, thereby decreasing the infiltration of autoreactive T cells to the eyes. In autoimmune diseases, cytokines and chemokines control the recruitment, survival, and expansion of autoreactive lymphocytes. Hence, blocking these chemokine receptors results in a reduction in T cell infiltration into the lesion in models of autoimmune diseases, such as EAU, experimental autoimmune encephalomyelitis (EAE) and alopecia areata. Indeed, a significant increase of chemokines, such as CCL2, CCL5, and CXCL10, was observed in the posterior segment of the eyes on day 14 after IRBP immunization in an EAU model. Similarly, the levels of CXCL9, CCL5, and CCL2 were significantly increased in the eyes of EAU mice. CXCL9/10/11 are ligands for CXCR3 which is mainly expressed in Th1 cells, and CXCR3-mediated chemotaxis is essential for the recruitment of Th1 cells to the sites where its ligands are secreted. Retina-reactive T cells also express CCR5, a receptor for CCL5, in EAU mice and rapidly migrated into the retina across the blood-retina barrier upon adoptive transfer. CCR2, a receptor for CCL2, is primarily associated with the recruitment of monocytes and macrophages to the eye with EAU, but CCR2-expressing T cells has also been found in lesions in autoimmune diseases. In addition, retinal antigens can directly function as tissue-specific chemoattractants and recruit retina-reactive T cells to the retina even in the absence of inflammation, which is mediated via CXCR3 and CXCR5 chemokine receptors. MSC-EVs inhibits the chemotaxis of retina-reactive T cells towards CXCL9, CCL2 and IRBP peptides. Consistent with in vivo observations, MC-EVs were more efficacious than ML-EVs in inhibiting the chemotaxis of T cells towards CXCL9, CCL2 and IRBP peptides. MSC-EVs suppressed T cell chemotaxis in response to CCL19, a ligand for CCR7 expressed on the surface of certain types of T cells including naïve T cells, central memory T cells and a subset of Tregs. As CCR7 plays a critical role in the migration of T cells to secondary lymphoid organs where they can interact with APCs and initiate an immune response, MSC-EVs can block the interaction between T cells and APCs in secondary lymphoid organs during the induction period of EAU before disease onset. Overall, targeting T cell chemotaxis is an effective approach for treatment of autoimmune diseases and in vitro chemotaxis assays with autoreactive T cells may be a useful biopotency assay for evaluating the therapeutic potency of MSC-EVs for the treatment of autoimmune diseases.

The biomarkers—TGF-β1, TSG-6, and/or let-7b-5p—contribute to the MoA of MSC-EVs in part. MSC-EVs suppressed the activation of MAPK/ERK signaling pathway in activated T cells with MC-EVs exhibiting higher potency than ML-EVs. Also, treatment with recombinant protein TGF-β1 or transient transfection with let-7b-5p mimics reproduced the effects of MSC-EVs on the MAPK/ERK pathway in IRBP-reactive T cells. The MAPK/ERK pathway is one of the major signaling pathways activated in cells upon stimulation with chemokines or other stimuli. This pathway is involved in cell migration by phosphorylating kinases, focal adhesion-associated proteins, microtubule-associated proteins or myosin light chain kinase. The inhibition of the MAPK/ERK pathway using pharmacological inhibitors or genetic manipulation directly reduces cell migration. Let-7b-5p targets different components of the MAPK/ERK pathway and regulates its activity. Similarly, TGF-β1 inhibits ERK phosphorylation, Ca²⁺ influx and NFATc translocation in anti-CD3/CD28-stimulated T cells. Moreover, TGF-β1 induces the expression of MAPK phosphatase MKP2 in a rapid manner, thereby inhibiting the phosphorylation of ERK in B lymphocytes within 1 h. Nevertheless, as MAPK/ERK signaling plays a central role in many cell responses including survival, proliferation, differentiation, migration and immune signaling, MSC-EVs can exhibit multiple effects on T cells by targeting MAPK/ERK signaling. Indeed, we previously demonstrated that MSC-EVs suppressed TCR signaling in splenocytes by inhibiting the translocation of P38 MAPK, NF-AT1 and P65 and the phosphorylation of LAT (Linker for activation of T cells). As provided herein, MSC-EVs increased apoptosis in retina-reactive T cells. Apoptosis plays an essential role in the control of immune response by eliminating target cells and activated lymphocytes. For instance, activated T cells increase the expression of Fas Ligand (FasL) and tumor necrosis factor-related apoptosis-inducing ligand (TRAIL), which in turn induces FasL- or TRAIL-mediated apoptosis in activated T cells and terminates the immune response. However, MAPK/ERK signaling is also activated by TCR stimulation and suppresses FasL- or TRAIL-mediated apoptosis in activated T cells, providing protective effects on T cells during the early phase of T cell activation. Therefore, the increased apoptosis of retina-reactive T cells by MSC-EVs can be attributed to the EV-mediated inhibition of MAPK/ERK signaling in T cells.

Administration of MSC-EVs immediately after IRBP immunization has a preventive effect on EAU development by inhibiting APC activation and Th1/Th17 development. Further, as provided herein, the therapeutic effect of MSC-EVs on EAU remains after the disease fully develops. Hence, MSC-EVs can be used not only for prevention before disease onset but also for the management of autoimmune disease after onset.

### EXAMPLES

The following examples are offered by way of illustration and not by way of limitation.

### Example 1: TGF-β1 Level in MSC-EVs Predicts the Immunomodulatory Potency of MSC-EVs In Vitro

The therapeutic effects of MSC-EVs in mice with ocular Sjögren's syndrome and LPS-induced endotoxemia were associated with their capacity to suppress Th1 and Th17 cytokines, such as IFN-γ, IL-2, TNF-α, IL-6 and IL-17, in splenocyte cultures upon TCR or TLR4 stimulation. Also, the immunomodulatory capacity of MSC-EVs in vivo and in vitro was significantly correlated with the expression levels of TGF-β1 and let-7b-5p in MSC-EVs. Additionally, similar to MSCs, EVs exhibited inter-donor differences in their potency. EVs from early passage MSCs showed higher immunomodulatory capacity than those from late passage MSCs, and microcarrier culture conditions of MSCs significantly improved the immunomodulatory potency of their EVs. Based on these findings, a method was developed using the key effectors, TGF-β1, TSG-6, and let-7b-5p as surrogate biomarkers for validating the immunomodulatory potency of MSC-EVs before in vivo administration.

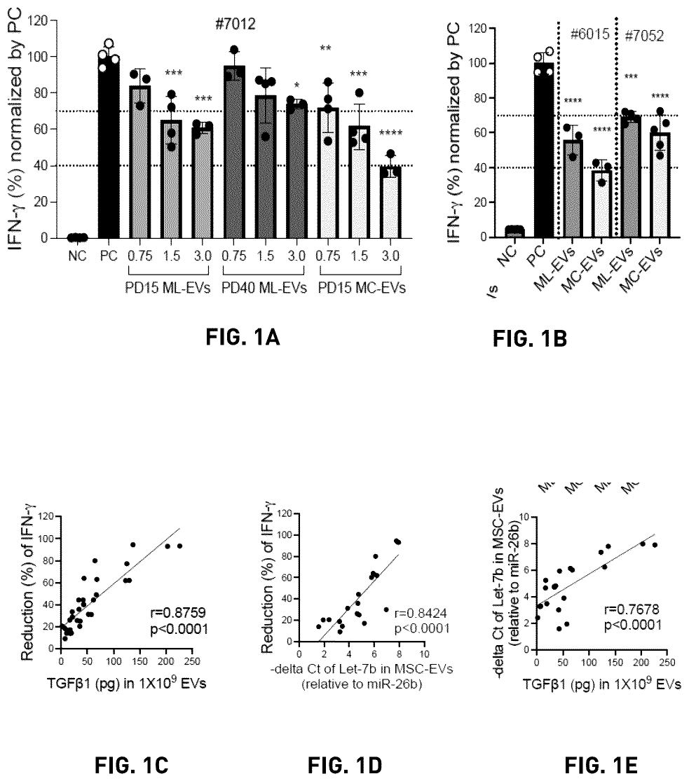

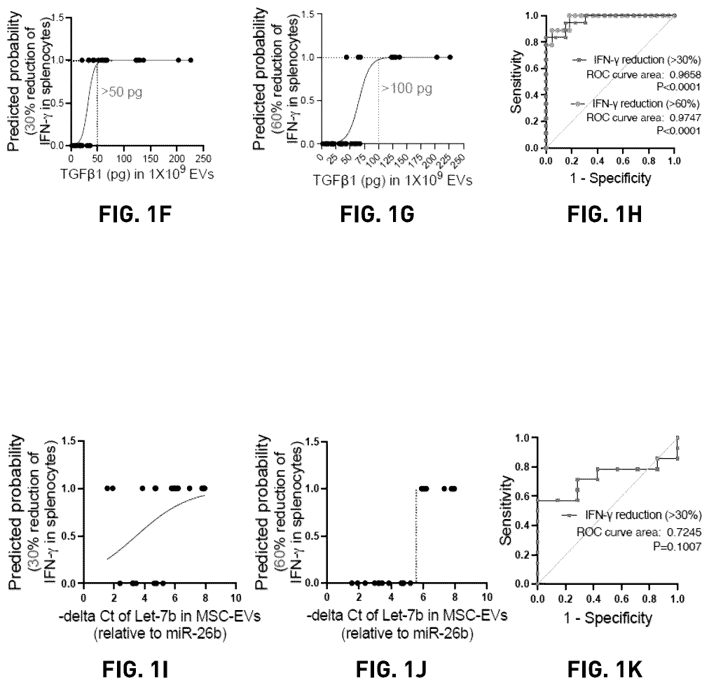

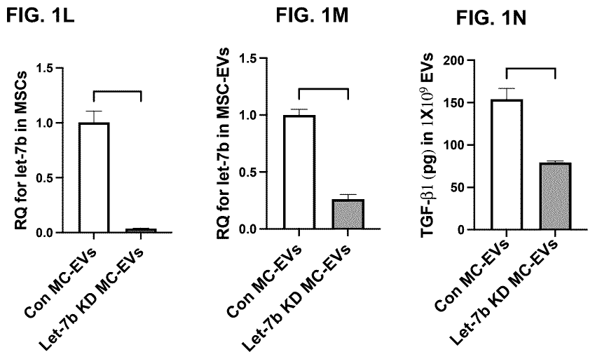

EVs were generated under various conditions (early- or late-passage MSCs, MSCs expanded under ML- or MC culture conditions, MSCs isolated from different donors) and their capacity to suppress IFN-γ secretion in anti-CD3/CD28-stimulated splenocyte cultures as well as the levels of TGF-β1 and let-7b-5p in MSC-EVs were examined. EVs from early-passage MSCs and from MSCs expanded under MC culture conditions were more efficacious (FIGS. 1A-1B). Inter-donor differences were observed in EVs from both ML- and MC-culture conditions (FIG. 1B). Next, a correlation between the levels of TGF-β1 and let-7b-5p in MSC-EVs generated under various conditions (early- or late-passage MSCs, monolayer or microcarrier culture conditions, different donors,) and their activity to suppress IFN-γ secretion in anti-CD3/CD28-stimulated splenocyte cultures were analyzed. The results revealed a significant correlation between the immunosuppressive potency of MSC-EVs and the levels of TGF-β1 and let-7b-5p (FIGS. 1C-1D). Moreover, the level of TGF-β1 in MSC-EVs was positively correlated with that of let-7b (FIG. 1E). Importantly, simple logistic regression analyses indicated that TGF-β1 and let-7b-5p levels in MSC-EVs predicted the efficacy of EVs in suppressing IFN-γ in anti-CD3/CD28-stimulated splenocytes (FIGS. 1F-1K). For example, 1×10⁹ EVs carrying >50 pg and 100 pg of TGF-β1 respectively suppressed 30% and 60% of IFN-γ in anti-CD3/CD28-stimulated splenocytes (FIGS. 1F to 1G). Similarly, let-7b-5p levels in EVs were able to discriminate EV potency to suppress 60% of IFN-γ secretion in anti-CD3/CD28-stimulated splenocytes with 100% sensitivity and 100% specificity (FIG. 1J). However, let-7b-5p was less useful in discriminating EVs with a low capacity to suppress IFN-γ secretion than TGF-β1 (FIG. 1I), as indicated by the ROC curve areas in FIG. 1K. Therefore, the data suggest that a simple ELISA assay, such as an ELISA assay for TGF-β1 or let-7b-5p in MSC-EVs can be used as a surrogate assay for evaluating the immunomodulatory potency of MSC-EVs.

When let-7b was knocked down in MSCs expanded on microcarriers (FIG. 1L), levels of let-7b decreased (FIG. 1M). Levels of TGF-β1 also decreased in MC-EVs (FIG. 1N). These results indicate that let-7b directly regulates TGF-b1 expression in MSC-EVs, thus, confirming the positive correlation between let-7b and TGF-b1 expression levels in MSC-EVs (refer to FIG. 1E).

### Example 2: MSC-EVs Carrying High Level of TGF-β1 Suppress EAU Progression in Mice

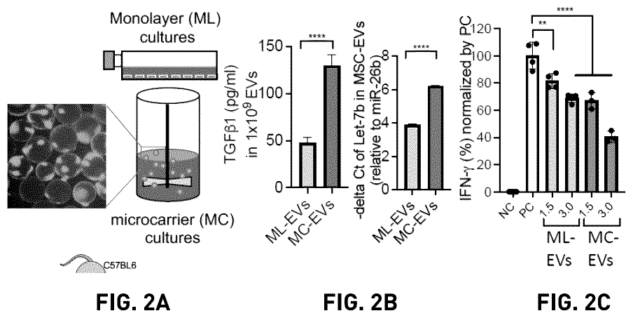

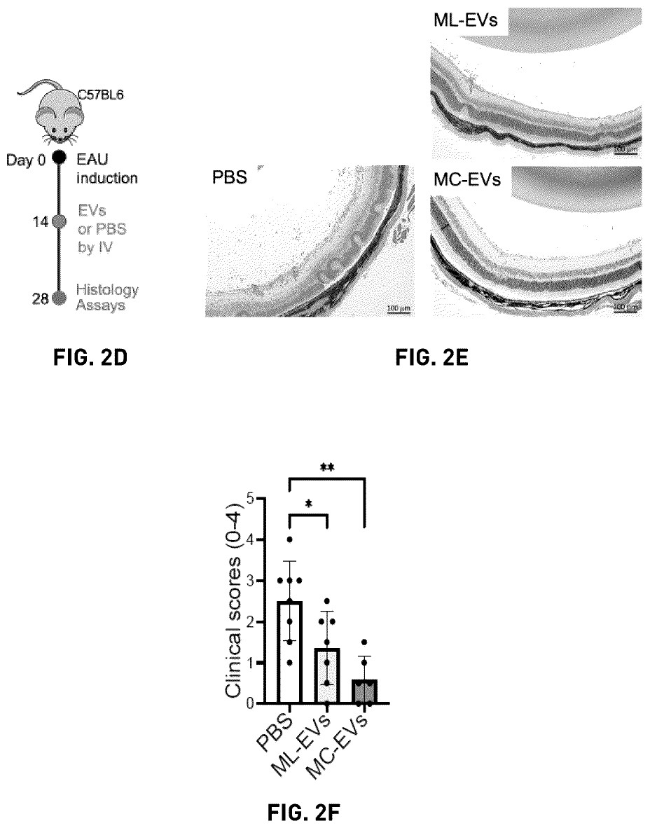

Next, the immunomodulatory effects of MSC-EVs carrying high levels of TGF-β1 was compared with those carrying low levels of TGF-β1 in a mouse model of EAU. To this end, EVs derived from MSCs in monolayer culture (ML-EVs) and EVs derived from MSCs in microcarrier culture (MC-EVs) were prepared (FIG. 2A). It was confirmed that MC-EVs contained >100 pg TGF-β1 as measured by ELISA while ML-EVs had about 50 pg TGF-β1 and that MC-EVs expressed higher levels of let-7b-5p than ML-EVs (FIG. 2B). Additionally, MSC-EVs' capacity to suppress IFN-γ secretion in anti-CD3/CD28-stimulated splenocytes isolated from naïve mice was validated (FIG. 2C). However, no significant differences were observed in EV particle size and the expression levels of EV markers among EVs.

The therapeutic effects of ML-EVs and MC-EVs were evaluated in mice with EAU wherein, upon IRBP immunization, retina-reactive Th1 and Th17 cells develop, migrate across the blood-retinal barrier, and cause cytotoxic destruction in the neural retina and related tissues. Mice were treated with an intravenous infusion of MSC-EVs on day 14 after IRBP immunization (FIG. 2D) when the disease manifests in the retina. As predicted by the surrogate assay, MC-EVs with higher TGF-β1 levels were more effective in protecting the retinal destruction than ML-EVs with lower TGF-β1 levels (FIGS. 2E-F). The retinal cross-sections showed severe disruption of retinal photoreceptor layer and infiltration of inflammatory cells in the retina and vitreous cavity in EAU mice treated with PBS. In contrast, there was little structural damage with few inflammatory infiltrates in the eyes of EAU mice that received MSC-EVs and ML-EVs, with MC-EVs being more effective than ML-EVs.

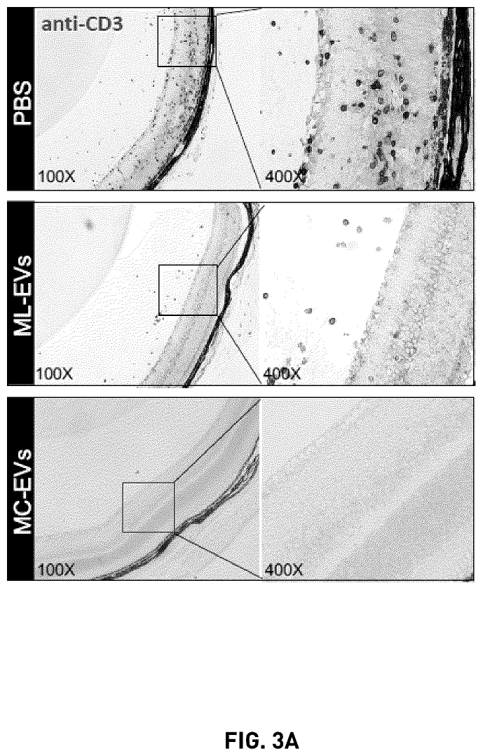

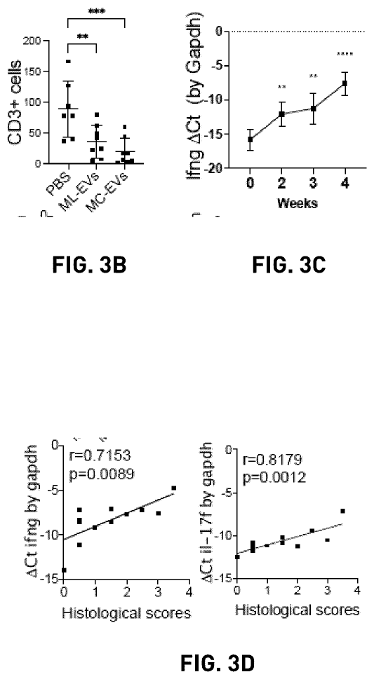

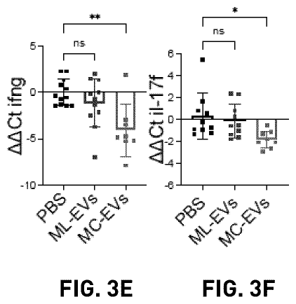

The effect of MSC-EVs in suppressing the infiltration of T cells in the retina tissues was examined. While IRBP immunization significantly increased CD3⁺ T cells and the expression levels of IFN-γ, a cytokine secreted from Th1 cells, in the eyes of EAU mice (FIGS. 3A-3C), both ML-EVs and MC-EVs significantly reduced the number of CD3⁺ T cells infiltrating the retina tissues (FIGS. 3A-3B). Similar to the results in the in vitro potency assay in FIG. 2C, MC-EVs were more efficacious than ML-EVs (FIG. 3B). Although histological analysis of retinal tissues is widely used to evaluate the disease severity of EAU in pre-clinical models, it is a labor-intensive and subjective assay. Therefore, quantitative readout assays to measure the severity of EAU in mice were developed. Histological scores of the right eyes of EAU or control mice were significantly correlated with the levels of IFN-γ and IL-17F mRNAs in the left eyes of the same mice (FIG. 3D), and the IFN-γ level was higher than the IL-17F level. These results were consistent with findings that the disease onset of EAU is associated with a mixed Th1/Th17 cytokine profile with IFN-γ predominating over IL-17 (Luger et al. 2008). Similar to the histological scores shown in FIG. 2F, the readout assays quantitatively assessed the therapeutic potency of MC-EVs in suppressing EAU severity in mice as compared to ML-EVs (FIG. 3E).

Collectively, the data indicate that MSC-EVs exerted therapeutic effects on EAU by inhibiting T cell infiltration into the retina, and EVs with higher TGF-β1 levels were more efficacious than those with lower TGF-β1 levels. Thus, these results support the notion that the measurement of TGF-β1 in MSC-EVs serves as a surrogate assay to predict the efficacy of MSC-EVs for the treatment of EAU.

### Example 3: MSC-EVs Reduce the Infiltration of Retinal Antigen-Reactive T Cells in Mice

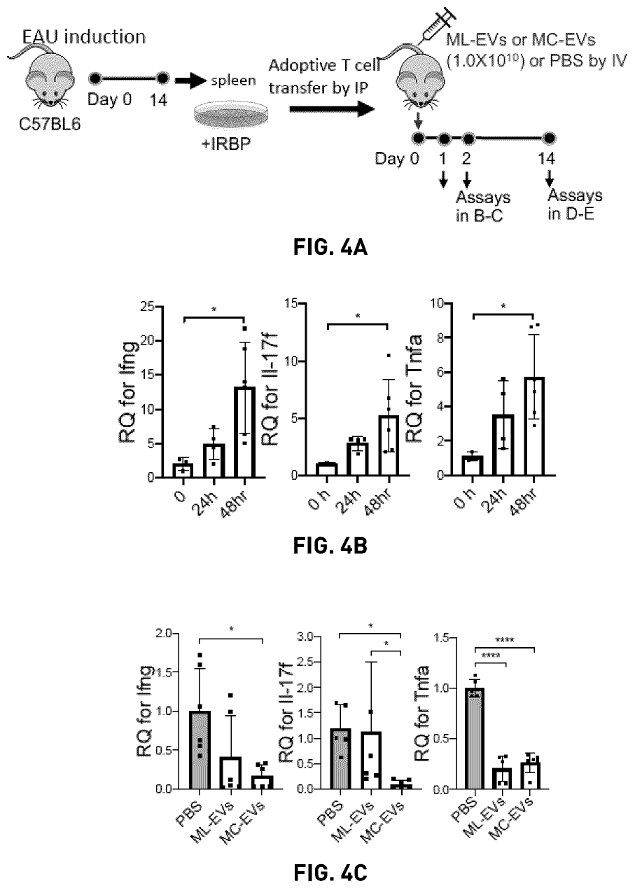

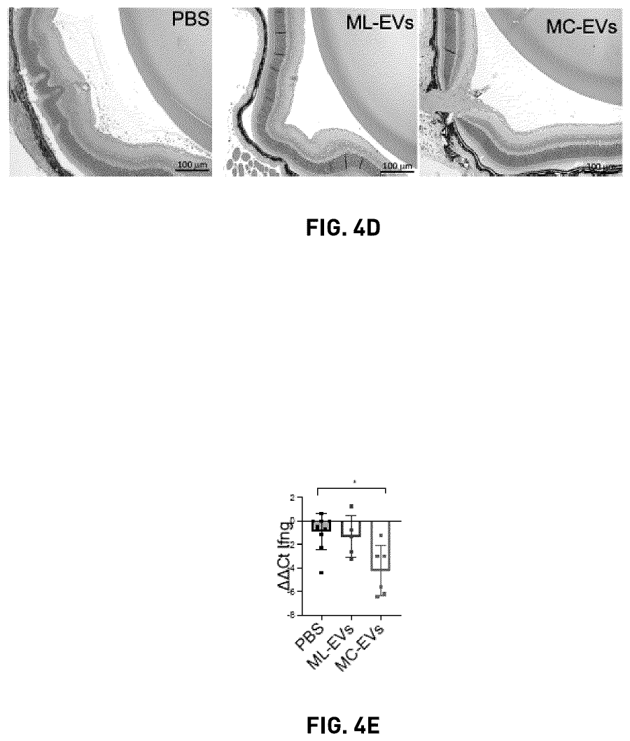

To understand the mechanism by which MSC-EVs halt disease progression in mice with established EAU, whether MSC-EVs directly suppress the infiltration of retinal antigen-reactive T cells in EAU mice was assessed. To this end, an adoptive transfer model of EAU in which the full histopathological changes are induced by adoptive transfer of retinal reactive T cells obtained from splenocytes of IRBP-immunized EAU mice was utilized. IRBP-reactive T cells were isolated from EAU mice and stimulated with IRBP and IL-12 for 3 days in vitro, and adoptively transferred them into naïve eight-week-old male C57BL/6J mice with or without MSC-EV treatment (FIG. 4A). Previous studies showed that upon adoptive transfer of IRBP-reactive T cells, they rapidly infiltrate into the eyes within 2 days and activate local myeloid cells, leading to massive cell entry into the eyes and severe retinal destruction on day 4 and 14, respectively (Lee, Richard W. et al. 2014, Prendergast et al. 1998). Consistent with the previous reports, a significant increase in IFN-γ and IL-17F mRNA levels as well as TNF-α in the eyes of recipient mice was observed on day 2 (FIG. 4B). Notably, the levels of IFN-γ and IL-17F as well as TNF-α were significantly decreased on day 2 in MC-EV-treated mice (FIG. 4C). Similarly, histological and molecular analyses on day 14 showed that MC-EVs significantly prevented EAU development (FIGS. 4D-4E). The mice treated with a single ML-EV injection exhibited a tendency towards reduced retinal destruction and IFN-γ levels, but it was not statistically significant (FIGS. 4D-4E).

Together, these results demonstrate that MSC-EVs halt the progression of EAU by inhibiting the infiltration of retinal antigen-reactive T cells. Additionally, the therapeutic potency of MSC-EVs was correlated with their TGF-β1 levels.

### Example 4: MSC-EVs Disrupt the Cell Cycle and Induce Apoptosis in Retinal Antigen-Reactive T Cells

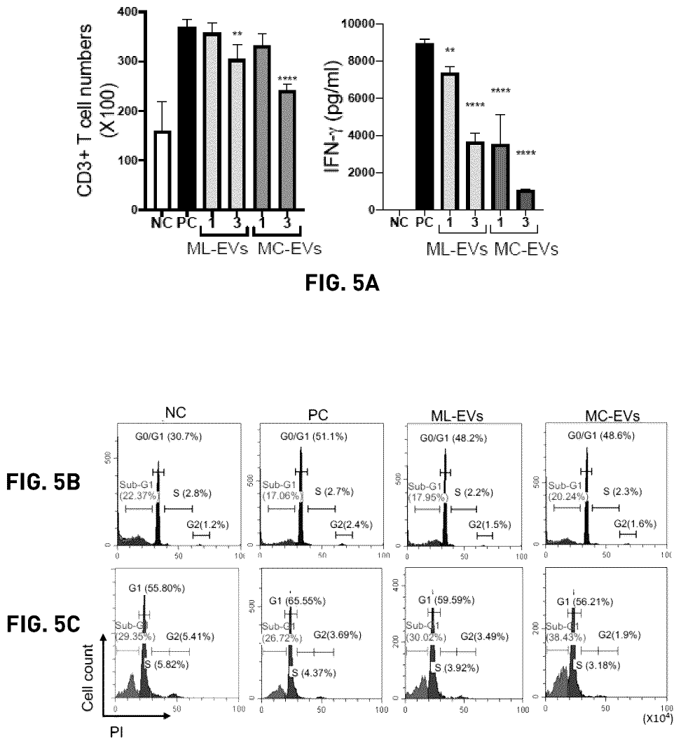

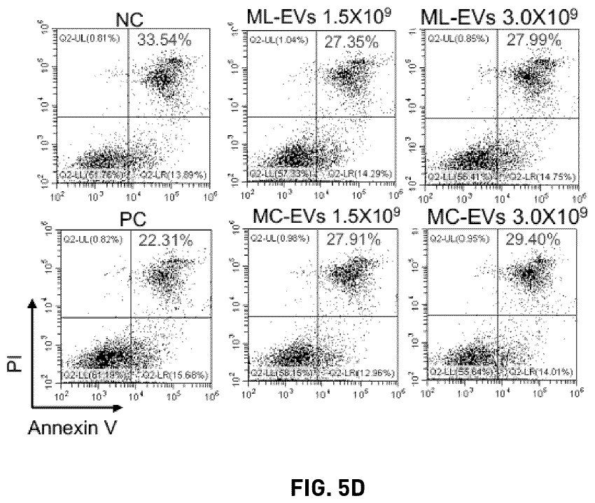

To investigate how MSC-EVs suppress the infiltration of IRBP-reactive T cells toward the eyes in recipient mice, we examined direct effects of MSC-EVs on the survival of IRBP-reactive T cells in vitro. An increase in the number of CD3⁺ T cells in splenocyte cultures derived from EAU mice upon IRBP stimulation, and MSC-EVs significantly suppressed the proliferation of CD3⁺ T cells (FIG. 5A). Also, MSC-EV treatment resulted in a decrease in the G2 and S phases of the cell cycle in IRBP-stimulated splenocytes while increasing the sub-G1 phase (FIG. 5B), indicating that MSC-EVs not only suppressed the cell cycle but also induced apoptosis in CD3⁺ T cells. The incubation of IRBP-reactive T cells with MSC-EVs for 24 hours showed similar effects observed in FIG. 5B (FIG. 5C). Additionally, MSC-EV treatment led to an increase in apoptosis (Annexin V+/PI+ cells) in IRBP-stimulated splenocytes (FIG. 5D) and overall, the effects of MC-EVs on inducing T cell cycle arrest and apoptosis were higher than those of ML-EVs (FIGS. 5B-5D). MSC-EVs suppress the cell cycle and induce apoptosis in T cells, thereby delaying disease progression in mice with EAU.

### Example 5: MSC-EVs Suppress the Migration of Retinal Antigen-Reactive T Cells Through Inhibition of MAPK/ERK Pathway

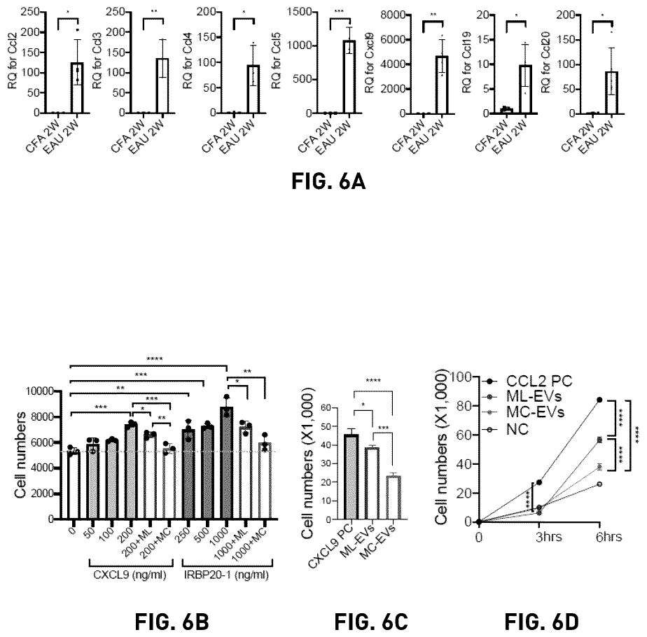

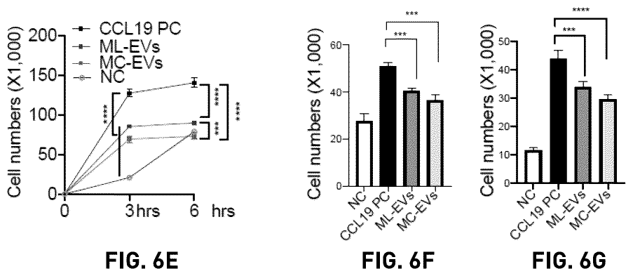

The inhibitory effects of MSC-EVs on the chemotaxis of IRBP-reactive T cells was examined in vitro. The levels of CXCL9, CCL5 and CCL2 were significantly increased in the eyes of EAU mice on day 14, as compared to control mice that received CFA alone (FIG. 6A). As autoantigens, such as IRBP, have been reported to induce CXCR3- and CXCR5-mediated chemotaxis in retinal reactive T cells (Howard et al. 2005), the chemotaxis of IRBP-reactive T cells toward CXCL9 or IRBP peptides was assessed in the presence or absence of MSC-EVs. The results showed that MC-EVs suppressed the migration of the T cells toward CXCL9 and IRBP peptides, and such effects were higher than ML-EVs (FIGS. 6B and 6C). Similar effects of ML- and MC-EVs were obtained with the chemotaxis of anti-CD3/CD28-stimulated T cells toward CCL2 or CCL19 (FIGS. 6D-6F) and with the chemotaxis of retinal antigen-reactive T cells toward CCL19 (FIG. 6G), which is associated with T cell and B cell migration to secondary lymphoid organs.

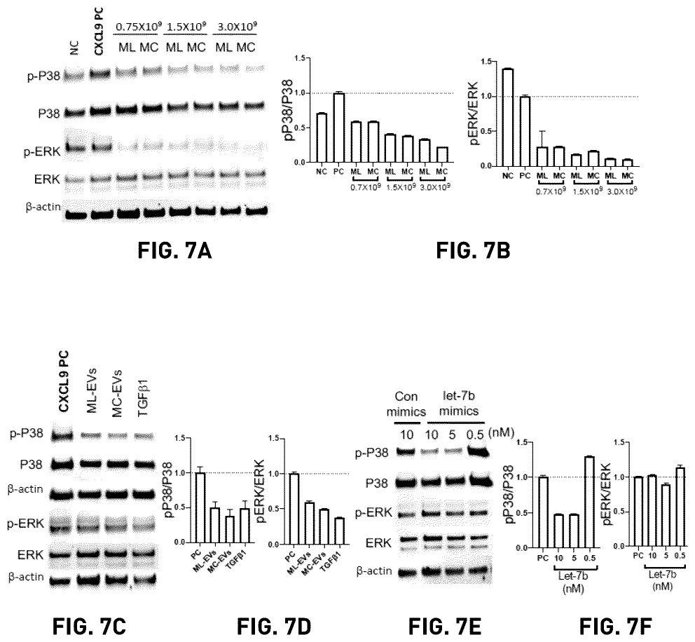

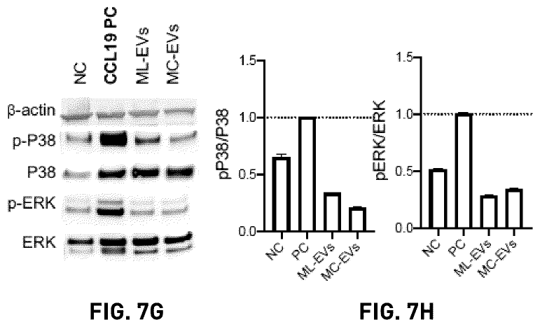

Furthermore, the effects of MSC-EVs on the P38 MAPK (mitogen-activated protein kinases) and ERK (extracellular signal-regulated kinase) singling pathway in T cells, which are among the major signaling pathways mediating cell migration (Tripathi and Poluri 2020), were evaluated. The results revealed that MSC-EVs suppressed the phosphorylation of P38 MAPK and ERK in naïve CD4⁺ T cells stimulated with CXCL9 (FIGS. 7A-7B). Both MC- and ML-EVs also suppressed the MAPK/ERK signaling pathway in IRBP-reactive T cells stimulated with CXCL9 (FIGS. 7C-7D) or CCL19 (FIGS. 7G-7H), with the effects of MC-EVs being greater than those of ML-EVs. As MC-EVs contained higher levels of TGF-β1 and let-7b-5p than ML-EVs, whether the inhibitory effect of MSC-EVs on the MAPK/ERK signaling pathway are mediated by TGF-β1 and let-7b-5p was evaluated. Indeed, 1 ng/ml of recombinant human (rh) TGF-β1, which is approximately twice the amount present in MC-EVs, showed a similar inhibitory effect on the phosphorylation of P38 MAPK in IRBP-reactive T cells (FIGS. 7C-7D). Moreover, transient transfection of let-7b-5p mimics into IRBP-reactive T cells rapidly suppressed the phosphorylation of P38 MAPK within 1 hours (FIGS. 7E-7F).

Collectively, the data indicate that MSC-EVs suppress T cell migration by inhibiting MAPK/ERK pathway, and the inhibitory effect of MSC-EVs on MAPK/ERK pathway in T cells can be mediated, at least in part, by TGF-β1, TSG-6, and/or let-7b-5p.

In summary, the results provided herein demonstrate a correlation between the immunosuppressive potency of MSC-EVs and the levels of TGF-β1 and/or let-7b-5p. Importantly, simple logistic regression analyses indicated that TGF-β1 levels in MSC-EVs predicted the efficacy of EVs in suppressing IFN-γ in anti-CD3/CD28-stimulated splenocytes. For example, 1×10⁹ EVs carrying greater than 50 pg and 100 pg of TGF-β1 respectively suppressed 30% and 60% of IFN-γ in anti-CD3/CD28-stimulated splenocytes. Furthermore, the immunomodulatory effects of MSC-EVs carrying high levels of TGF-β1 were validated in a mouse model of experimental autoimmune uveitis (EAU).

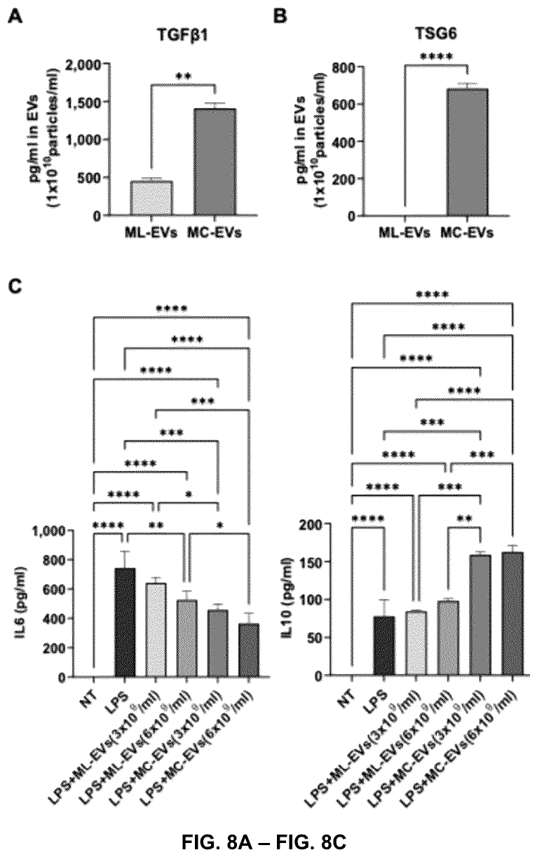

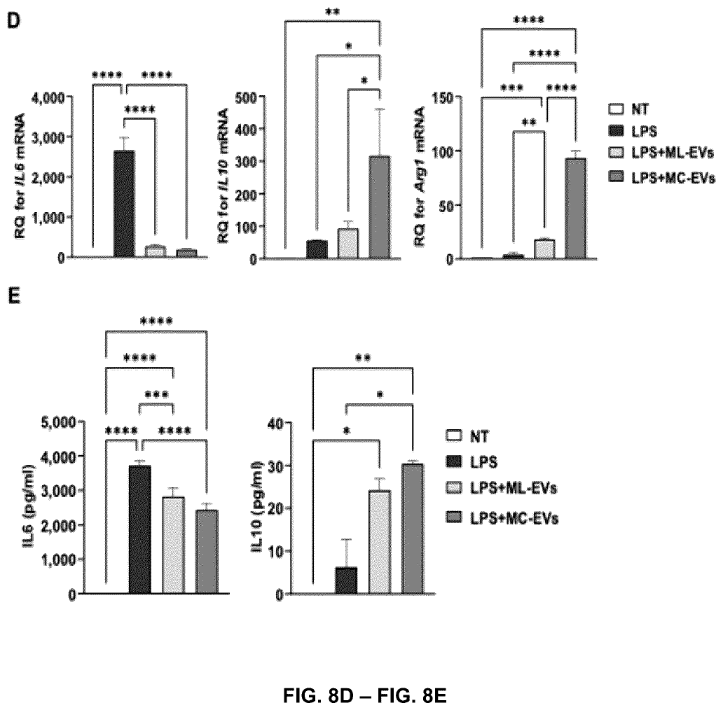

The MC culture conditions significantly increased TNF-α-stimulated gene 6 (TSG-6) protein expression in MSC-EVs. TSG-6 is known for its anti-inflammatory effects, modulating macrophage plasticity, and inhibiting neutrophil infiltration. Therefore, the levels of the key anti-inflammatory proteins, TGFβ1 and TSG-6, were assessed in ML-EVs and MC-EVs using ELISA. FIGS. 8A-8E provide a comparison of anti-inflammatory effects in MSC-EVs from monolayer (ML) and microcarrier (MC) Cultures. FIGS. 8A and 8B are graphical representations of the results from ELISAs for TGFβ1 and TSG-6 proteins in ML-EVs and MC-EVs. FIG. 8C is a graphical representations of the anti-inflammatory response of ML-EVs and MC-EVs in LPS-stimulated macrophages. Murine RAW 264.7 macrophage cells were stimulated with a dose of 10 ng/mL LPS with or without MSC-EVs. Cells or conditioned media were assayed at 6 and 24 hours after stimulation. The TGF-β1 levels in MC-EVs were more than three times higher than in ML-EVs (FIG. 8A). In addition, TSG-6 was undetectable in ML-EVs but was present at significantly higher levels in MC-EVs (>50 pg in 1×10⁹ EVs; FIG. 8B). Next, the anti-inflammatory response of these MSC-EVs were evaluated in lipopolysaccharide (LPS)-stimulated RAW 264.7 macrophages. Macrophages were treated with LPS with or without the addition of either ML-EVs or MC-EVs, and inflammatory markers were assessed at 6 and 24 hours after stimulation. FIG. 8C is a graphical representation of the assessment of IL-6 (left) and IL-10 (right) secretion in conditioned media 6 hours after stimulation and treatment with ML-EVs or MC-EVs at concentrations of 3×10⁹/mL or 6×10⁹/mL by ELISA. At 6 hours, both ML-EVs and MC-EVs had a significant reduction in the secretion of IL-6 and enhancement of IL-10 secretion, with MC-EVs demonstrating a more pronounced effect (FIG. 8C). The gene expression changes in the macrophages were further analyzed at 24 hours post-stimulation. FIGS. 8D and 8E are graphical representations of the assessment of anti-inflammatory markers in macrophages 24 hours after stimulation and treatment with ML-EVs or MC-EVs at a concentration of 3×10⁹/mL. FIG. 8D is a graphical representation of the qPCR analysis of the gene expression of IL-6, IL-10, and Arg1. FIG. 8E is a graphical representation of the IL-6 and IL-10 secretion in conditioned media by ELISA. The qPCR results showed that both ML-EVs and MC-EVs had an increased expression of the anti-inflammatory markers IL10 and Arg1, and a suppression of the pro-inflammatory IL-6 gene expression. Although ML-EVs showed an increase in IL10 expression compared to the LPS-only group, the increase was not statistically significant (FIG. 8D). Furthermore, at 24 hours, IL-6 and IL-10 levels were measured in the cell supernatant using ELISA. Both ML-EVs and MC-EVs had reduced secretion of IL-6 compared to the LPS-only group. The IL-10 levels increased with statistical significance only in the MC-EVs group (FIG. 8E).

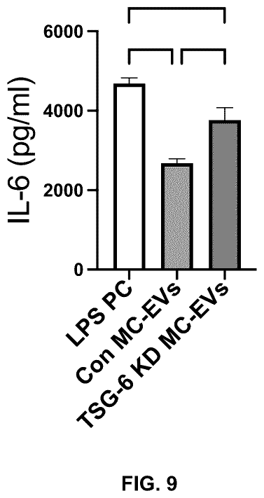

To determine TSG-6 as a key effector in MC-EVs, TSG-6 in MC-EVs was knockdowned by engineering MSCs using TSG-6 siRNAs and their anti-inflammatory potency was assessed. the TSG-6 KD MC-EVs were less effective in suppressing the secretion of IL-6 than control MC-EVs. FIG. 9 is a graphical representation of the comparison of anti-inflammatory effects in MC-EVs vs TSG-6 KD MC-EVs. Murine RAW 264.7 macrophage cells were stimulated with a dose of 10 ng/mL LPS with or without MSC-EVs (3×10⁹/mL). Assessment of IL-6 secretion in conditioned media 6 hours after stimulation by ELISA. TSG-6 was shown as one of the key effectors responsible for EV-mediated anti-inflammatory activities. Therefore, measurements of both TGF-b1 and TSG-6 can be used as a surrogate assay to validate EV anti-inflammatory potency.

These data indicate that the therapeutic potency of EVs can be evaluated by measuring the amount of one or more of TGF-β1, TSG-6, and let-7b-5p in the EVs. In certain embodiments, the method includes measuring the amount of TGF-β1 or TSG-6 in the EVs and in other embodiments, the method includes measuring the amount of TGF-β1 and TSG-6 in the EVs.

### Materials and Methods

MSC-EV preparations and Characterization. Human bone marrow-derived MSCs (donor #7012, #6015 and #7052) were obtained from the Institute for Regenerative Medicine at Texas A&M University, expanded with a low-density seeding method as described previously (Lee, R. H. et al. 2014). MSC-EVs were isolated as we have shown previously (Kim et al. 2020). Briefly, passage 2 (P2) human MSCs were plated at 200 cells/cm2 in the cell stack with complete culture medium (CCM) containing 17% FBS. After the cells reached about 70% confluency, MSCs were incubated with a chemically defined and protein free (CDPF) medium prepared using Chinese hamster ovary cells (CD-CHO medium, Invitrogen, Carlsbad, CA, USA) for EV production. The media was discarded after 6 hours and replaced by fresh CDPF medium. After 48 hours incubation, the media was recovered for isolating EVs. For MC-EVs, P2 human MSCs (1×10⁶ cells) were seeded to 1 g of low-concentration Synthemax II microcarriers (Corning Life Sciences, NY, USA) with a volume of 25 mL of CCM containing 5% fetal bovine serum (FBS) in a 125-mL spinner flask for the 24 h. Twenty-five mL CCM was added to cells to make a final volume of 50 mL for the remaining time according to the manufacturer's protocol. Cultures were incubated at 37° C. with agitation at 30 rpm for 5 min every 4 h. Half of the volume medium was replenished every 2 days. On day 7, medium was removed, microcarriers were washed in PBS twice, and 50 mL of CDPF medium was added. After 24 h, the medium was recovered for EV collection. The CDPF media recovered from MSC cultures was filtered with 0.22 μm filter to remove cellular debris, concentrated by Vivaspin tubes (10 kDa molecular weight cutoff, GE Healthcare, Chicago, IL, USA) and ultracentrifuged at 100,000×g for 16 hours at 4° C. using a Sorvall WX Floor Ultra Centrifuge with an AH-629 36-mL swinging bucket rotor (Thermo Fisher Scientific, Waltham, MA, USA). EV pellets were recovered and resuspended in cold PBS. As a part of characterization, the particle size and number of EVs were analyzed using the NanoSight LM 10 nanoparticle tracking analysis system (Malvern, Malvern, UK). The expression levels of EV surface markers CD9, CD63 and CD81 were analyzed by flow cytometry (CytoFLEX, Beckman coulter) using magnetic beads coated with anti-CD63 (human CD63 Isolation/Detection kit; Invitrogen) with anti-CD63-FITC (clone H5C6; BD Biosciences), anti-CD81-PE (clone JS-81; Biosciences) and anti-CD9-PE (Clone M-L13; BD Biosciences). Western blot assays were also performed on isolated EVs using antibodies against CD63 (MX-49.129.5; Santa Cruz Biotechnology, Dallas, TX), TSG101 (JJ0900; Invitrogen), APOA1 (Invitrogen) and Calnexin (C5C9; Cell signaling, Danvers, MA). Further, the isolated EVs were assayed for TGF-β1 and let-7b-5p expression levels using ELISA and RT-PCR, as described previously (Kim et al. 2020) and stored at −80° C. until used for in vitro splenocyte assays, and in vivo studies.

EAU mouse model. The experimental protocols were approved by the Institutional Animal Care and Use Committee of the Texas A&M University. Six to eight-week-old C57BL/6J mice (The Jackson Laboratory, Bar Harbor, ME, USA) were immunized with subcutaneous (SC) injection into tail base and tights of the retina-specific antigen, interphotoreceptor retinal binding protein (IRBP) peptide 1-20, GPTHLFQPSLVLDMAKVLLD (500 μg; New England Peptide, Inc, Gardner, MA, USA) emulsified in complete Freund adjuvant (BD Diagnostic Systems) containing Mycobacterium tuberculosis (2.5 mg/ml; BD Difco, Franklin Lakes, NJ). Simultaneously, the mice received intraperitoneal injection (IP) of 0.5 μg pertussis toxin (300 μl; List Biologicals, Campbell, CA). On day 14 or 17 after immunization, MSC-EVs (10×10⁹ particles/mouse) or PBS were injected via tail vein into the mice. To induce EAU in B10.RIII mice (The Jackson Laboratory; 6- to 8-week-old male and female), 25 μg of IRBP peptide 161-180 (SGIPYIISYLHPGNTILHVD) emulsified in complete Freund adjuvant (BD Diagnostic Systems) containing Mycobacterium tuberculosis (2.5 mg/ml; BD Difco) were used as described previously (Agarwal et al. 2012). For an adoptive transfer model of EAU, splenocytes were isolated from EAU mice at day 14 after IRBP immunization and stimulated with medium containing IRBP peptides (20 mg/ml) and IL-12 (5 ng/ml) for 3 days as described previously (Agarwal et al. 2012). After 72 hours of culture, T cells were collected using Lympholyte M (Accurate Biochemicals) and transferred into naïve C57BL/6J mice (4×10⁷/mouse; via IP injection). After 15 minutes of IRBP-reactive T cell transfer, mice received MSC-EVs (10×10⁹ particles/mouse) or PBS via tail vein. MSC-EVs validated with human TGF-β1 ELISA (R&D systems) were used in in vivo studies.

Histology. The mice were sacrificed, and eyeballs were collected for assays. Eyeballs were subjected to histological and molecular assays. For histology, the eyeballs were fixed in 4% Glutaraldehyde for 1 h, transferred into 10% phosphate-buffered formalin at least overnight and embedded in paraffin. Serial 5 μm thick sections were cut and stained with H-E and CD3 immunohistochemical staining. For CD3 immunohistochemical staining, a rabbit anti-mouse CD3 (cat #ab5690, Abcam, Cambridge, MA) was used as a primary antibody. The pathologic features of the retina were examined, and histological disease score was assessed in a blinded manner on a scale of 0 to 4 using the criteria previously defined by Caspi (Agarwal et al. 2012). The number of CD3-stained cells was calculated under a microscope using×20 object.

Isolation and activation of T cells. CD4⁺ T cells were isolated from splenocytes from BALB/c mice by CD4⁺ T Cell Isolation Kit II (Miltenyi Biotec, San Diego, CA) according to the manufacture's protocol. The CD4⁺ T cells were cultured in 96-well plates with CD3/CD28 beads (Life Technologies) with or without EVs in RPMI-1640 medium containing 5% heat-inactivated FBS, 100 units/ml penicillin, 100 mg/ml streptomycin and 0.05 nM 2-Mercaptoethanol. Two or three days later, the levels of Th1 cytokines were detected by ELISA (R&D systems) according to the manufacture's protocols.

Real-time RT-PCR assay. For molecular assays, the eyeballs were lysed in RNA isolation reagent (RNA Bee; Tel-Test, Friendswood, TX) and homogenized using a sonicator (Ultrasonic Processor; Cole Parmer Instruments, Vernon Hills, IL). Total RNA was extracted from the eyeballs or splenocyte culture using RNeasy Mini kit (Qiagen, Valencia, CA), and double-stranded cDNA were synthesized by reverse transcription (High Capacity RNA-to-cDNA Kit; Applied Biosystems; Life Technologies). Real-time PCR amplification (ABI 7900 Sequence Detector; Applied Biosystems) was performed using TaqMan Universal PCR Master Mix (Applied Biosystems). PCR probe and primer sets were purchased from Applied Biosystems (TaqMan Gene Expression Assay): Tnf-α, Il-1β, Il-2, Il-4, Il-10, Il-6, Il-12a, Il-17a, and Ifn-γ. For relative quantitation of gene expression, mouse-specific Gapdh primers and probe (Mm99999915_g1) were used.

Analysis for cell cycle and apoptosis. Splenocytes from EAU mice were isolated and cultured with IRBP 161-180 peptides (20 mg/ml) with or without MSC-EVs (1.5 to 3×10⁹/ml) for 2 days. Floating cells were collected, and analyzed for cell cycle and cell death using a flow cytometry with commercial kits (FxCycle™ PI/RNase Staining Solution, Thermo Fisher Scientific; Annexin V-FITC Apoptosis Detection Kit, Sigma-Aldrich, St. Louis, MO).

T-cell migration assay. Mouse CD4+ T cells were isolated from spleen of naïve C57/BL6 mice using a commercial CD4+ T Cell Isolation Kit (Miltenyi Biotec, Germany) as per manufacturer's protocol. To activate T cells, 1 to 1.5×10⁶ purified T cells/mL were added in RPMI culture medium containing 10% FBS, Dynabeads® magnetic beads anti-CD3/CD28 (Thermo Fisher Scientific) at a bead-to-cell ratio of 1:1 and 30 U/mL recombinant mouse IL-2 (R&D systems, Minneapolis, MN) and incubated for 2-3 days in a humidified CO2 incubator at 37° C. Also, splenocytes were isolated from C57/BL6 mice with EAU on day 14 after EAU induction and expanded with RPMI culture medium containing 10% FBS, IRBP peptides (20 mg/ml) and IL-12 (5 ng/ml, R&D systems) for 3 days (Agarwal et al. 2012). The top wells were loaded with stimulated T cells (2.5×10⁵) from naïve C57BL/6 mice or EAU mice and the lower chambers contained recombinant mouse CCL2 (20 ng/ml, R&D systems), CXCL9 (50 to 200 ng/ml, R&D systems), IRBP (250 to 1 000 ng/ml) or CCL19 (20 ng/ml, R&D systems). After 3 or 6-hour incubation at 37° C., the cells that migrated to the lower chamber were counted.

Western Blot assays. Total protein was isolated from naïve CD4+ T cells (5×10⁵) or IRBP-reactive T cells (5×10⁵) stimulated with CXCL9 (200 ng/ml) or CCL9 (20 ng/ml) with/without MSC-EVs (0.75 to 3×10⁹ particles/ml) or human recombinant TGF-β1 (R&D systems) for 1 hour. IRBP-reactive T cells were transfected with control scrambled miRNA mimics or let-7b-5p-5p-5p-5p-5p-5p mimics (Thermo Fisher Scientific) using RNAiMAX (Thermo Fisher Scientific) and simultaneously treated with CXCL9 (200 ng/ml) for 1 h. The following antibodies were used: anti-RAS, anti-p38, anti-p-P38, anti-p-ERK and anti-ERK (Cell signaling, Danvers, MA), and anti-β-actin (Invitrogen).

Statistical Analysis. All data were analyzed using One-way ANOVA followed by Dunnett's or Tukey's multiple comparison tests or Student t-test. Statistical analysis and graphical generation of data were done with GraphPad Prism software (GraphPad, San Diego, CA, USA).

When ranges are disclosed herein, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, reference to values stated in ranges includes each and every value within that range, even though not explicitly recited. Thus, every point or individual value may serve as its own lower or upper limit combined with any other point or individual value or any other lower or upper limit, to recite a range not explicitly recited.

Other objects, features and advantages of the disclosure will become apparent from the foregoing drawings, detailed description, and examples. These drawings, detailed description, and examples, while indicating specific embodiments of the disclosure, are given by way of illustration only and are not meant to be limiting. In further embodiments, features from specific embodiments may be combined with features from other embodiments. For example, features from one embodiment may be combined with features from any of the other embodiments. In further embodiments, additional features may be added to the specific embodiments described herein. It should be understood that although the disclosure contains certain aspects, embodiments, and optional features, modification, improvement, or variation of such aspects, embodiments, and optional features can be resorted to by those skilled in the art, and that such modification, improvement, or variation is considered to be within the scope of this disclosure.

What is claimed is:

1. A method for evaluating therapeutic potency of extracellular vesicles (EVs), the method comprising measuring amount of one or more of TGF-β1 (Transforming Growth Factor-β1), TSG-6 (Tumor Necrosis Factor-Stimulated Gene 6 protein), and let-7b-5p in the EVs.

2. The method of claim 1, comprising measuring the amount of TGF-β1 and TSG-6 in the EVs.

3. The method of claim 1, wherein the EVs are derived from mesenchymal stem cells.

4. The method of claim 1, wherein the EVs are derived from cells in monolayer culture or microcarrier culture or spheroid culture.

5. A method for selecting a population of extracellular vesicles (EVs) for treatment of a subject, the method comprising:
measuring amount of one or more of TGF-β1 (Transforming Growth Factor-β1), TSG-6 (Tumor Necrosis Factor-Stimulated Gene 6 protein), and let-7b-5p in the population of EVs; and
in response to the measured amount of the one or more of TGF-β1, TSG-6, and let-7b-5p in the population of EVs being above a predetermined threshold, selecting the population of EVs for treatment of a subject.

6. The method of claim 5, comprising measuring the amount of one or more of TGF-β1, TSG-6, and let-7b-5p in the population of EVs by ELISA and/or RT-PCR.

7. The method of claim 5, wherein the predetermined threshold is 50 pg of TGF-β1 in 1×10⁹ EVs.

8. The method of claim 5, wherein the predetermined threshold is 10 pg of TSG-6 in 1×10⁹ EVs.

9. The method of claim 5, wherein the population of EVs are derived from mesenchymal stem cells in monolayer culture or microcarrier culture or spheroid culture.

10. The method of claim 5, wherein the subject has an autoimmune disease or an inflammatory disease.

11. A method for treating a disease in a subject, the method comprising:
measuring amount of one or more of TGF-β1 (Transforming Growth Factor-β1), TSG-6 (Tumor Necrosis Factor-Stimulated Gene 6 protein), and let-7b-5p in the extracellular vesicles (EVs); and
isolating a portion of the EVs containing the one or more of TGF-β1, TSG-6, and let-7b-5p above a predetermined threshold; and
administering a therapeutically effective amount of the portion of the EVs to the subject, thereby treating the disease.

12. The method of claim 11, comprising measuring amount of TGF-β1 and TSG-6 in the EVs.

13. The method of claim 11, wherein the predetermined threshold is 50 pg of TGF-β1 in 1×10⁹ EVs.

14. The method of claim 11, wherein the predetermined threshold is 100 pg of TGF-β1 in 1×10⁹ EVs.

15. The method of claim 11, wherein the predetermined threshold is 10 pg of TSG-6 in 1×10⁹ EVs.

16. The method of claim 11, wherein the predetermined threshold is 50 pg of TSG-6 in 1×10⁹ EVs.

17. The method of claim 11, wherein the EVs are derived from mesenchymal stem cells.

18. The method of claim 11, wherein the EVs are derived from cells in monolayer culture or microcarrier culture or spheroid culture.

19. The method of claim 11, wherein the disease is an immune-mediated disease.

20. The method of claim 11, wherein the disease is an inflammatory disease.
